(19) United States
(12) Patent Application Publication
(10) Pub. No.: US 2005/0137567 A1
(43) Pub. Date: Jun. 23, 2005

(54) For introducing medicinal fluids from multiple sources into a mixing chamber and transmission and retention into an ambulatory container

(76) Inventor: David Almendarez, Houston, TX (US)

Correspondence Address: William C. Norvell, Jr.;BEIRNE, MAYNARD & PARSON, L.L.P., First Interstate Tower, 25th Floor, 1300 Post Oak Boulevard, Houston, TX, 77056-3000, US

(21) Appl. No.: 10/738,166

(22) Filed: Dec. 17, 2003

(51) Int. Cl. A61M 31/00

(52) U.S. Cl. 604/500; 604/82

(57) ABSTRACT

A method is disclosed for introducing medicinal fluids from multiple sources into a mixing chamber and for transmission and retention into an ambulatory container having a transmission conduit including a one way check valve assembly. The mixing and the transmission through the valve of the fluids includes application and removal of occlusion and differential pressure forces during mixing and transmission.

### BACKGROUND OF THE INVENTION

(1) Field of the Invention

The invention relates to the controlled mixing of a plurality of medicinal fluids in a mixing chamber and a fluid line into an ambulatory container or bag for subsequent introduction into a patient through intravenous injection using the bag as a container for the medicinal injections.

(2) Brief Description of the Prior Art

In hospitals and clinics, it is frequently necessary to provide solutions for intravenous administration to a patient which contains a variety of drugs, or medicinal fluids, in a single solution container. A common example of such a need arises when a patient is receiving all of his nutritional needs intravenously. In this situation, the patient will typically receive a basic solution containing amino acids, dextrose, and fat emulsions which provide a major portion of the patient's nutritional needs. However, this solution is sometimes insufficient to maintain a patient for an extended period of time. Therefore, a typical total parenteral solution includes as many as eight to twelve, or more, additional additives. The additives are typically minute quantities of vitamins, minerals, electrolytes, and the like.

In recent years, ambulatory containers, or bags, suspended and controlled through comparatively small mobile pump units that can be moved to locations proximate the bed or chair for a patient have been used to contain the mixed fluids, not only for nutritional purposes, but also for administering liquid pain killers and other commonly injected medicinal fluids from the bag, through a conduit which is inserted into a blood vessel in the patient's arm or leg. Typical of such pump units well known to those skilled in the art is the I PUMP (trademark of Baxter Healthcare Corporation, Deerfield, Ill.) pain management system. This product is commercially available from a number of convenient health care products vendors throughout the United States.

Typical of the ambulatory containers or bags in commercial use is the single use sterilized 250 ml. Drug Bag, Product No. 2L3257, of Baxter Healthcare Corporation of Deerfield, Ill. This bag may be used in the practice of the present method. Prior to the present invention, such bags were filled with medicinal fluids using a very time consuming procedure which included the incorporation of a peristaltic pump and injector system, such as the BAXA REPEATER, trademarked product of the Baxa Corporation, Englewood, Co., Product No. H 938-099-1. U.S. Pat. No. 5,024,347 and U.S. Pat. No. 4,976,590 each disclose such a peristaltic pump system. Each fluid is introduced and measured into the pump and injector system separately, one after another, a task that was obviously very time consuming.

I have discovered that the above-described problems associated with prior ambulatory bag filling equipment and procedures is greatly remedied by providing a method which incorporates the use of ambulatory bag which includes a fluid transmission conduit and a check valve assembly which are selectively removable from the bag subsequent to completion of the mixing procedure wherein the mixing procedure is continuous during the preparation period within the transmission line upstream of the check valve assembly as well as in a mixing chamber within a device that further uses a combination of occlusion and pressure differential forces, all as herein described. By such method, the ambulatory bag may be filled much faster than by use of conventional peristaltic pump methods.

### SUMMARY OF THE INVENTION

The present invention is directed to a method for introducing medicinal fluids from more than one source into a mixing chamber and thereafter transmission and retention into an ambulatory bag or container. In the practice of the method, the ambulatory bag which is used is expandable and has first and second opposite ends. The first end has a smooth outer surface means thereon for suspending the bag during introduction of a measured amount of the medicinal fluids into the bag. The second end of the bag has an opening there through and into the bag for selective receipt of a removable fluid transfer line. The line includes a one-way check valve having upstream and downstream faces permitting the medicinal fluids to be introduced within the bag in a first fluid flow direction through the line and preventing removal of the fluids within the bag through the line and the check valve immediate the downstream face in a second flow direction.

A mixing chamber is provided which is in fluid communication between the source container and the ambulatory container. Each source container has a separable fluid communication conduit connected to the mixing chamber which is initially subject to a removable occlusion force. The fluid transmission line communicates between the mixing chamber and the ambulatory container and is initially also subject to a removable occlusion force.

An amount of at least one of the fluids to be transferred and retained within the ambulatory container is selected. Thereafter, a signal indicating the amount of fluids to be transferred is generated. Next, at least one of the selected fluids is transferred from the source container to the mixing chamber under the influence of a pressure differential and removal of the occlusion force between the source containers and the mixing chamber to thereby mix the medicinal fluids within the mixing chamber and within the fluid communication line immediate the upstream face of the check valve.

The amount of the fluid in the mixing chamber is measured and then another signal is generated which is indicative of the actual amount of the selected fluid present in the chamber. Fluid transfer is terminated when the actual amount of the fluid in the chamber is equal to or greater than the selected amount by again subjecting the fluid communication between one of the sources containers and the mixing chamber and the ambulatory container to an occlusion force.

Thereafter, the fluid is transferred from the mixing chamber to the transmission line and the ambulatory container under a pressure differential and the removal of the occlusion force between the mixing chamber, the transmission line and the ambulatory container. Finally, the fluid transmission line and the check valve are removed from the opening in the second end of the ambulatory bag and the opening in the bag is sealingly blocked.

### DESCRIPTION OF THE PREFERRED EMBODIMENTS

The present method incorporates usage of an ambulatory bag or container. An ambulatory container or bag, as opposed to other medicinal containers or bags, is one that is intended to be suspended over or otherwise above a physically mobile pump system and which uses gravity to permit a controlled and measurable fluid flow from the bag into the mobile pump and thence into the patient through intravenous or other method of continuous injection. Many such containers which may be used in the practice of the present invention may be used, but I presently prefer to use a Baxter 250 ml Drug Bag, Product No. 2L3257, manufactured by Baxter Healthcare Corporation of Deerfield, Ill. It is readily and commercially available from a number of source selling pharmacy and hospital care products.

At one end of the bag is a short fluid conduit or transmission line for introduction and discharge of fluids. It is initially capped at its outer end. The cap is removed and a fluid line having two ends is used for connection to the chort fluid conduit extending into the bag. A line which may be conveniently used is part of a container assembly containing multi ports and is introduced through one of the ports. The assembly is marketed commercially by Baxter Healthcare Corporation of Deerfield, Ill., under the trademark VIAFLEX, 250 ml capacity. The conduit may simply be cut from the container at the opening. The other or opposite end of the conduit incorporates a socket for easy manual insertion and removal from a port extending to the mixing chamber. To the other end is affixed a one way check valve having upstream and downstream faces permitting the medicinal fluids to be introduced within the bag in a first flow direction through the line from the chamber immediate the upstream face and preventing removal of the medicinal fluids within the bag through the line and the check valve immediate the downstream face in a second flow direction. Typical of such check valves is the Burron normally closed back check valve made and sold by Burron Medical, Inc., Bethlehem, Pa. This product is disclosed in detail in U.S. Pat. No. 4,535,820, entitled “Normally Closed Check Valve”, issued Aug. 20, 1985. The valve has a body composed of two cylindrical containers which compliment each other. Each of the containers has a tubular projection with a liquid opening there through. One of the cylindrical chambers is provided with a plurality of longitudinally extending ribs for preventing sticking of a valve disc contained in the interior of the housing. A transverse bar is also contained just downstream of the disc for pressing against the disc. The other body component is provided with a pointed triangular support and also a plurality of radially extending ribs for preventing the valve disc from opening so far as to close off the egress port provided with the second element. A rubber resilient valve disc is retained between the pointed triangular support and the transverse bar when the valve unit is assembled by sonic welding into a permanently assembled device. The end of the above described conduit or transmission line opposite the socket is secured around the upstream outer side of the check valve assembly, and a the nipple or short end of conduit extending out of the ambulatory container or bag is inserted within an protruding extension on the downstream side of the valve assembly. The bag, transmission line and check valve assembly are now ready to be secured just upwardly or over a mixing chamber having occlusion force removal and pressure differential features.

The chamber and peristaltic pump for mixing the medicinal composition for use in the present invention may be as Disclosed in U.S. Pat. No. 5,056,568, entitled “Automated System for Adding Multiple Fluids to a Single Container”, issued Oct. 15, 1991. Commercially, the described product is made and sold as the AUTOMIX 3+3 Compounder, by Clintec Nutrition Company, Deerfield, Ill. This device accurately transfers multiple individual fluids from multiple source containers for transmission into the medicinal container. Fluid flows from the multiple source containers through individual fluid inlet conduits into a measuring chamber having a single fluid outlet conduit in fluid communication with the single receiving container. The chamber also has a pressure conduit. A first occlusion means for selectively preventing fluid flow from each of the individual fluid inlet conduits to the chamber is provided. Pressure means and differential pressure source for selectively creating positive and negative pressures in the chamber to control the rate of fluid flow through the chamber is also provided. In order to control fluid flow from the chamber to the receiving container, a second occlusion means for selectively occluding fluid flow from the chamber outlet fluid conduit to the receiving chamber is provided.

Control means for controlling the first and second occlusion means and the pressure means is provided to perform various functions. For example, the control means causes the first occlusion means to allow fluid to flow through at least one of the individual fluid conduits while causing the second occlusion means to prevent fluid flow into the receiving container. The control means also simultaneously causes the pressure means to create a negative pressure in the chamber to precisely control the amount and rate of fluid flow into the chamber. The control means further causes the first occlusion means to prevent fluid flow through all of the individual fluid conduits after a predetermined amount of fluid has been delivered to the chamber. The control means then further causes the second occlusion means to allow fluid to flow from the receiving chamber through the outlet conduit while simultaneously causing the pressure means to create a positive pressure in the chamber to force fluid from the chamber into the receiving container.

After the ambulatory bag and transmission line with check valve in place have been prepared, the bag is placed just slightly above or over the mixing chamber/pump assembly and the socket at one end of the transmission line is placed into the unit on the discharge side. One or more medicinal fluids are selected for transmission into the mixing chamber and the appropriate amount of each is measured using the chamber/pump assembly. An electronic signal is generated by the mixing/pump assembly which is indicative of the medicinal fluids to be present in the mixing chamber. Thereafter, transfer of the fluids is terminated by application of an occlusion force. The fluids are then mixed in conventional fashion, such as through agitation of the mixing chamber. The mixed fluid is then transferred to the transmission line and the ambulatory container through the one way check valve through pressure differential while the occlusion force is removed from within the mixing chamber, the transmission line and the ambulatory container. The transmission line with the check valve is then removed from the opening as the bag is rotated so that the opening faces upwardly to prevent discharge of fluids in the bag by application of gravitational force. The opening may then be sealed and blocked by the application of a closure cap to the short conduit or nipple portion extending from the bag.

Although the invention has been described in terms of specified embodiments which are set forth in detail, it should be understood that this is by illustration only that the invention is not necessarily limited thereto, since alternative embodiments and operating techniques will become apparent to those skilled in the art in view of the disclosure. Accordingly, modifications are contemplated which can be made without departing from the spirit of the described invention.

1) A method for introducing medicinal fluids from multiple sources into a mixing chamber and transmission and retention into an ambulatory container, comprising the steps of:
(a) providing an ambulatory container in the form of an expandable bag having first and second opposite ends, said first end having a smooth outer surface means thereon for suspending said bag during introduction of a measured amount of said medicinal fluids into said bag, said second end having an opening there through and into said bad and selectively receiving a removable fluid transfer line including a one-way check valve having upstream and downstream faces permitting the medicinal fluids to be introduced within the bag in a first flow direction through the line from the chamber immediate said upstream face and preventing removal of the medicinal fluids within the bag through the line and the check valve immediate the downstream face in a second flow direction;
(b) providing a mixing chamber in fluid communication between said source containers and said ambulatory container, each source container having a separable fluid communication conduit connected to said mixing chamber which is initially subject to a removable occlusion force, said fluid transmission line communicating between said mixing chamber and said ambulatory container and initially subject to a removable occulsion force;
(c) selecting an amount of at least one of said fluids to be transferred and retained within said ambulatory container;
(d) generating a signal indicating said amount of said fluids to be transferred;
(e) transferring at least one of said selected fluids from its source container to said mixing chamber under the influence of a pressure differential and by removing the occulsion force between said source containers and said mixing chamber to thereby mix said medicinal fluids within the mixing chamber and within the fluid communication line immediate the upstream face of the check valve;
(f) measuring the amount of fluid in the mixing chamber;
(g) generating a signal indicative of the actual amount of selected fluid present in said chamber;
(h) terminating transfer of said fluid when said actual amount of said fluid present in said chamber is equal to or greater than said selected amount by again subjecting said fluid communication between at least one of said source containers and said mixing chamber and said ambulatory container to an occulsion force;
(i) transferring said fluid from said mixing chamber to said transmission line and said ambulatory container under the influence of a pressure differential and by removing the occulsion force between the mixing chamber, the transmission line and the ambulatory container; and
(j) removing the transmission line and the check valve from the opening in the second end of the ambulatory bag and sealingly blocking said opening.
